(19) United States
(12) Patent Application Publication
(10) Pub. No.: US 2025/0025198 A1
(43) Pub. Date: Jan. 23, 2025

(54) SURGICAL INSTRUMENT WITH INTEGRATED TISSUE STOP

(71) Applicant: Cilag GmbH International, Zug (CH)

(72) Inventor: William D. Dannaher, Cincinnati, OH (US)

(21) Appl. No.: 18/415,073

(22) Filed: Jan. 17, 2024

Related U.S. Application Data
(60) Provisional application No. 63/514,011, filed on Jul. 17, 2023.

(51) Int. Cl. A61B 17/32; A61B 17/00

(52) U.S. Cl. CPC A61B 17/320068; A61B 2017/00982; A61B 2017/320074

(57) ABSTRACT

A surgical instrument includes an end effector located at a distal end of a shaft and is selectively movable between an open configuration and a closed configuration. The end effector includes a guide with an ultrasonic blade extending therefrom and a clamp arm having a clamp pad and movable relative to the ultrasonic blade. The end effector further includes a tissue stop encircling the guide and having a leading surface. The tissue stop is configured to prevent tissue from moving proximally past the clamp pad during a closure stroke. In some versions, a leading surface of the tissue stop prevents tissue from moving proximally. The tissue stop may include a pair of deflectable flaps having the leading surface, wherein the deflectable flaps abut the clamp arm or other features of the end effector as the closure stroke is performed.

### PRIORITY

This application claims priority to U.S. Provisional Pat. App. No. 63/514,011, entitled “Methods of End Effector Assembly and Related Arrangements for Surgical Instruments,” filed Jul. 17, 2023, the entirety of which is hereby incorporated by reference herein.

### BACKGROUND

A variety of surgical instruments include an end effector having a blade element that vibrates at ultrasonic frequencies to cut and/or seal tissue (e.g., by denaturing proteins in tissue cells). These instruments include piezoelectric elements that convert electrical power into ultrasonic vibrations, which are communicated along an acoustic waveguide to the blade element. The precision of cutting and coagulation may be controlled by the surgeon's technique and adjusting the power level, blade edge, tissue traction and blade pressure.

Examples of ultrasonic surgical instruments include the HARMONIC ACE® Ultrasonic Shears, the HARMONIC WAVER Ultrasonic Shears, the HARMONIC FOCUS® Ultrasonic Shears, and the HARMONIC SYNERGY® Ultrasonic Blades, all by Ethicon Endo-Surgery, Inc. of Cincinnati, Ohio. Further examples of such devices and related concepts are disclosed in U.S. Pat. No. 5,322,055, entitled “Clamp Coagulator/Cutting System for Ultrasonic Surgical Instruments,” issued Jun. 21, 1994, the disclosure of which is incorporated by reference herein; U.S. Pat. No. 5,873,873, entitled “Ultrasonic Clamp Coagulator Apparatus Having Improved Clamp Mechanism,” issued Feb. 23, 1999, the disclosure of which is incorporated by reference herein; U.S. Pat. No. 5,980,510, entitled “Ultrasonic Clamp Coagulator Apparatus Having Improved Clamp Arm Pivot Mount,” filed Oct. 10, 1997, the disclosure of which is incorporated by reference herein; U.S. Pat. No. 6,325,811, entitled “Blades with Functional Balance Asymmetries for use with Ultrasonic Surgical Instruments,” issued Dec. 4, 2001, the disclosure of which is incorporated by reference herein; U.S. Pat. No. 6,773,444, entitled “Blades with Functional Balance Asymmetries for Use with Ultrasonic Surgical Instruments,” issued Aug. 10, 2004, the disclosure of which is incorporated by reference herein; and U.S. Pat. No. 6,783,524, entitled “Robotic Surgical Tool with Ultrasound Cauterizing and Cutting Instrument,” issued Aug. 31, 2004, the disclosure of which is incorporated by reference herein.

Still further examples of ultrasonic surgical instruments are disclosed in U.S. Pub. No. 2006/0079874, entitled “Tissue Pad for Use with an Ultrasonic Surgical Instrument,” published Apr. 13, 2006, the disclosure of which is incorporated by reference herein; U.S. Pub. No. 2007/0191713, entitled “Ultrasonic Device for Cutting and Coagulating,” published Aug. 16, 2007, the disclosure of which is incorporated by reference herein; U.S. Pub. No. 2007/0282333, entitled “Ultrasonic Waveguide and Blade,” published Dec. 6, 2007, the disclosure of which is incorporated by reference herein; U.S. Pub. No. 2008/0200940, entitled “Ultrasonic Device for Cutting and Coagulating,” published Aug. 21, 2008, the disclosure of which is incorporated by reference herein; U.S. Pub. No. 2009/0105750, entitled “Ergonomic Surgical Instruments,” published Apr. 23, 2009, the disclosure of which is incorporated by reference herein; U.S. Pub. No. 2010/0069940, entitled “Ultrasonic Device for Fingertip Control,” published Mar. 18, 2010, the disclosure of which is incorporated by reference herein; and U.S. Pub. No. 2011/0015660, entitled “Rotating Transducer Mount for Ultrasonic Surgical Instruments,” published Jan. 20, 2011, the disclosure of which is incorporated by reference herein; and U.S. Pub. No. 2012/0029546, entitled “Ultrasonic Surgical Instrument Blades,” published Feb. 2, 2012, the disclosure of which is incorporated by reference herein.

Some of ultrasonic surgical instruments may include a cordless transducer such as that disclosed in U.S. Pub. No. 2012/0112687, entitled “Recharge System for Medical Devices,” published May 10, 2012, the disclosure of which is incorporated by reference herein; U.S. Pub. No. 2012/0116265, entitled “Surgical Instrument with Charging Devices,” published May 10, 2012, the disclosure of which is incorporated by reference herein; and/or U.S. Pat. App. No. 61,410,603, filed Nov. 5, 2010, entitled “Energy-Based Surgical Instruments,” the disclosure of which is incorporated by reference herein.

Additionally, some ultrasonic surgical instruments may include an articulating shaft section. Examples of such ultrasonic surgical instruments are disclosed in U.S. Pub. No. 2014/0005701, entitled “Surgical Instruments with Articulating Shafts,” published Jan. 2, 2014, the disclosure of which is incorporated by reference herein; and U.S. Pub. No. 2014/0114334, entitled “Flexible Harmonic Waveguides/Blades for Surgical Instruments,” published Apr. 24, 2014, the disclosure of which is incorporated by reference herein.

While several surgical instruments and systems have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an exemplary surgical instrument having one example of an end effector;

FIG. 2 depicts a side elevational view of the end effector of the instrument of FIG. 1, in a closed configuration;

FIG. 3 depicts a perspective view of the end effector of FIG. 2, in an open configuration;

FIG. 4 depicts a perspective view of the end effector of FIG. 2, in a closed configuration;

FIG. 5 depicts a side elevational view of another example of an end effector for incorporation into the instrument of FIG. 1, in a closed configuration;

FIG. 6 depicts a side elevational view of the end effector of FIG. 5 with a first exemplary tissue stop applied thereto, in a closed configuration;

FIG. 7 depicts a perspective view of the tissue stop of FIG. 6;

FIG. 8 depicts a cross-sectional view of the tissue stop of FIG. 6 taken along line8-8 of FIG. 7 with a guide extending therethrough;

FIG. 9 depicts a side elevational view of the end effector of FIG. 5 with a second exemplary tissue stop applied thereto, in an open configuration;

FIG. 10 depicts a side elevational view of the end effector of FIG. 5 with the second tissue stop of FIG. 9 applied thereto, in a closed configuration;

FIG. 11 depicts a front elevational view of the end effector of FIG. 5 with an exemplary deflectable flap of the second tissue stop of FIG. 9 abutting an exemplary clamp arm of the end effector; and

FIG. 12 depicts a perspective view of the second tissue stop of FIG. 9 showing an exemplary interior chamfered area, an exemplary series of cut-outs, and an exemplary fluid associated with the deflectable flap of FIG. 11 of the second tissue stop of FIG. 10.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument.

### I. Exemplary Ultrasonic Surgical Instrument

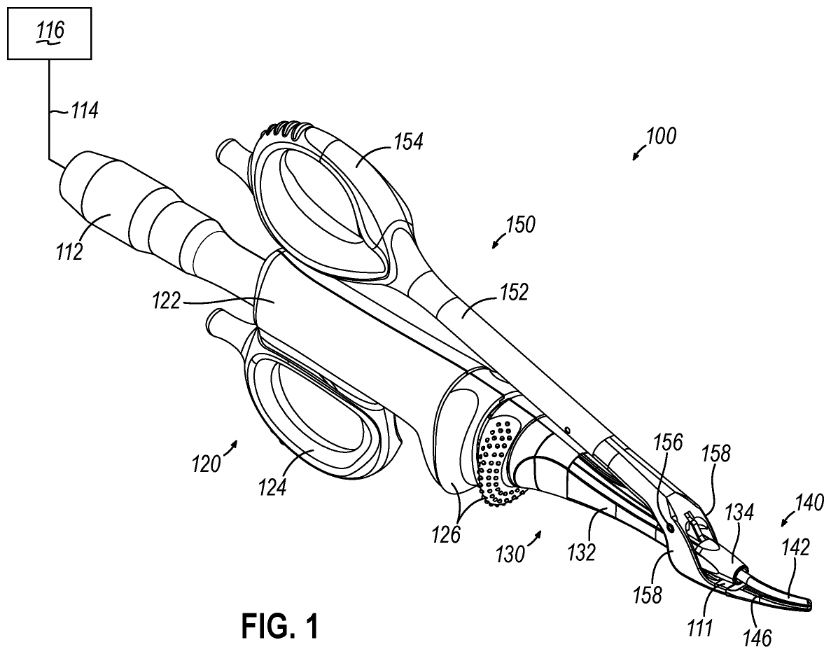

FIGS. 1-4 illustrate an exemplary ultrasonic surgical instrument (100). At least part of each instrument (100) may be constructed and operable in accordance with at least some of the teachings of U.S. Pat. Nos. 5,322,055; 5,873,873; 5,980,510; 6,325,811; 6,773,444; 6,783,524; U.S. Pub. No. 2006/0079874; U.S. Pub. No. 2007/0191713; U.S. Pub. No. 2007/0282333; U.S. Pub. No. 2008/0200940; U.S. Pub. No. 2009/0105750; U.S. Pub. No. 2010/0069940; U.S. Pub. No. 2011/0015660; U.S. Pub. No. 2012/0112687; U.S. Pub. No. 2012/0116265; U.S. Pub. No. 2014/0005701; U.S. Pub. No. 2014/0114334; U.S. Pat. App. No. 61,410,603; and/or U.S. patent application Ser. No. 14,028,717. The disclosures of each of the foregoing patents, publications, and applications are incorporated by reference herein. As described therein and as will be described in greater detail below, instrument (100) is operable to cut tissue and seal or weld tissue (e.g., a blood vessel, etc.) substantially simultaneously. It should also be understood that instrument (100) may have various structural and functional similarities with the HARMONIC ACER Ultrasonic Shears, the HARMONIC WAVE® Ultrasonic Shears, the HARMONIC FOCUS® Ultrasonic Shears, and/or the HARMONIC SYNERGY® Ultrasonic Blades. Furthermore, instrument (100) may have various structural and functional similarities with the devices taught in any of the other references that are cited and incorporated by reference herein.

To the extent that there is some degree of overlap between the teachings of the references cited herein, the HARMONIC ACER Ultrasonic Shears, the HARMONIC WAVE® Ultrasonic Shears, the HARMONIC FOCUS® Ultrasonic Shears, and/or the HARMONIC SYNERGY® Ultrasonic Blades, and the following teachings relating to instruments (100), there is no intent for any of the description herein to be presumed as admitted prior art. Several teachings herein will in fact go beyond the scope of the teachings of the references cited herein and the HARMONIC ACER Ultrasonic Shears, the HARMONIC WAVER Ultrasonic Shears, the HARMONIC FOCUS® Ultrasonic Shears, and the HARMONIC SYNERGY® Ultrasonic Blades.

FIG. 1 illustrates an exemplary ultrasonic surgical instrument (100) that is configured to be used in open surgical procedures. Instrument (100) of this example comprises a handle assembly (120), a shaft assembly (130), and an end effector (140). Handle assembly (120) comprises a body (122) including a finger grip ring (124) and a pair of buttons (126). Instrument (100) also includes a clamp arm assembly (150) that is pivotable toward and away from body (122). Clamp arm assembly (150) includes a shank (152) with a thumb grip ring (154). Thumb grip ring (154) and finger grip ring (124) together provide a scissor grip type of configuration. It should be understood, however, that various other suitable configurations may be used, including but not limited to a pistol grip configuration.

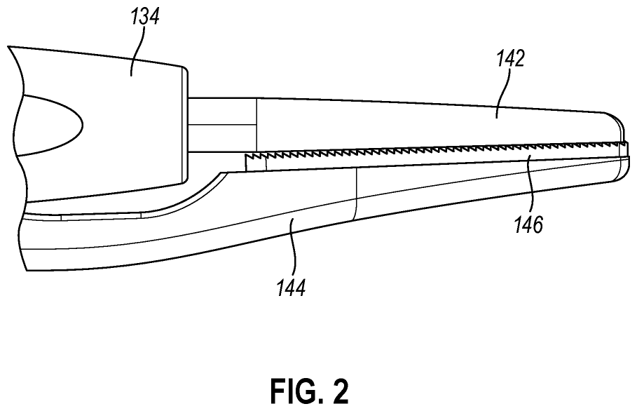

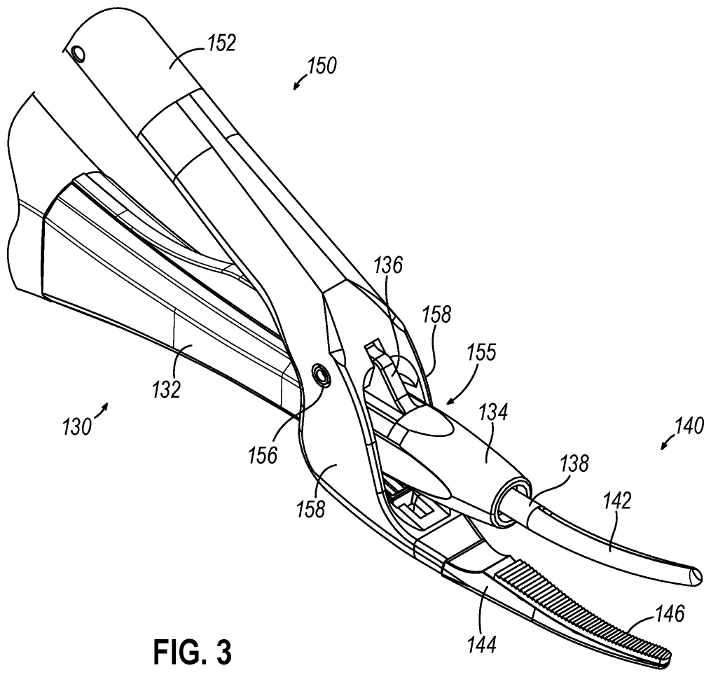

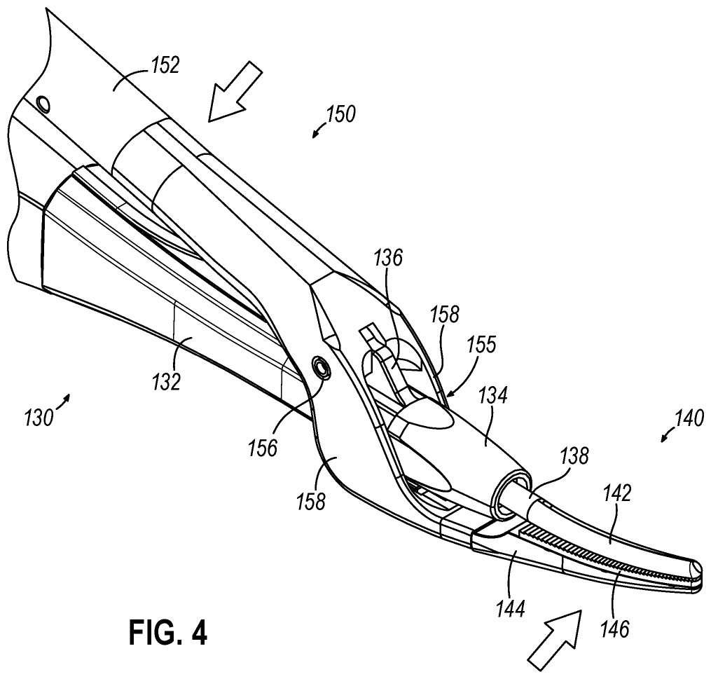

Shaft assembly (130) comprises an outer sheath (132) extending distally from body (122). A cap (134) is secured to the distal end of sheath (132). As best seen in FIGS. 2-4, end effector (140) comprises an ultrasonic blade (142) and a clamp arm (144). Ultrasonic blade (142) extends distally from cap (134). Clamp arm (144) is an integral feature of clamp arm assembly (150). Clamp arm (144) includes a clamp pad (146) facing ultrasonic blade (142). Clamp arm assembly (150) is pivotally coupled with outer sheath (132) via a pin (156). Clamp arm (144) is positioned distal to pin (156); while shank (152) and thumb grip ring (154) are positioned proximal to pin (156). Thus, as shown in FIGS. 3-4, clamp arm (144) is pivotable toward and away from ultrasonic blade (142) based on pivoting of thumb grip ring (154) toward and away from body (122) of handle assembly (120). It should therefore be understood that an operator may squeeze thumb grip ring (154) toward body (122) to thereby clamp tissue between clamp pad (146) and ultrasonic blade (142) to transect and/or seal the tissue. In some versions, one or more resilient members are used to bias clamp arm (144) to the open configuration shown in FIG. 3. By way of example only, such a resilient member may comprise a leaf spring, a torsion spring, and/or any other suitable kind of resilient member.

Referring back to FIG. 1, an ultrasonic transducer assembly (112) extends proximally from body (122) of handle assembly (120). Transducer assembly (112) is coupled with a generator (116) via a cable (114). Transducer assembly (112) receives electrical power from generator (116) and converts that power into ultrasonic vibrations through piezoelectric principles. Generator (116) may include a power source and control module that is configured to provide a power profile to transducer assembly (112) that is particularly suited for the generation of ultrasonic vibrations through transducer assembly (112). By way of example only, generator (116) may comprise a GEN 300 sold by Ethicon Endo-Surgery, Inc. of Cincinnati, Ohio. In addition, or in the alternative, generator (116) may be constructed in accordance with at least some of the teachings of U.S. Pub. No. 2011/0087212, entitled “Surgical Generator for Ultrasonic and Electrosurgical Devices,” published Apr. 14, 2011, the disclosure of which is incorporated by reference herein. It should also be understood that at least some of the functionality of generator (116) may be integrated into handle assembly (120), and that handle assembly (120) may even include a battery or other on-board power source such that cable (114) is omitted. Still other suitable forms that generator (116) may take, as well as various features and operabilities that generator (116) may provide, will be apparent to those of ordinary skill in the art in view of the teachings herein.

Ultrasonic vibrations that are generated by transducer assembly (112) are communicated along an acoustic waveguide (138), which extends through shaft assembly (130) to reach ultrasonic blade (142). Waveguide (138) is secured within shaft assembly (130) via a pin (not shown), which passes through waveguide (138) and shaft assembly (130). This pin is located at a position along the length of waveguide (138) corresponding to a node associated with resonant ultrasonic vibrations communicated through waveguide (138). As noted above, when ultrasonic blade (142) is in an activated state (i.e., vibrating ultrasonically), ultrasonic blade (142) is operable to effectively cut through and seal tissue, particularly when the tissue is being clamped between clamp pad (146) and ultrasonic blade (142). It should be understood that waveguide (138) may be configured to amplify mechanical vibrations transmitted through waveguide (138). Furthermore, waveguide (138) may include features operable to control the gain of the longitudinal vibrations along waveguide (138) and/or features to tune waveguide (138) to the resonant frequency of the system.

In the present example, the distal end of ultrasonic blade (142) is located at a position corresponding to an anti-node associated with resonant ultrasonic vibrations communicated through waveguide (138), in order to tune the acoustic assembly to a preferred resonant frequency fₒ when the acoustic assembly is not loaded by tissue. When transducer assembly (112) is energized, the distal end of ultrasonic blade (142) is configured to move longitudinally in the range of, for example, approximately 10 to 500 microns peak-to-peak, and in some instances in the range of about 20 to about 200 microns at a predetermined vibratory frequency fₒ of, for example, 55.5 kHz. When transducer assembly (112) of the present example is activated, these mechanical oscillations are transmitted through the waveguide to reach ultrasonic blade (102), thereby providing oscillation of ultrasonic blade (102) at the resonant ultrasonic frequency. Thus, when tissue is secured between ultrasonic blade (142) and clamp pad (46), the ultrasonic oscillation of ultrasonic blade (142) may simultaneously sever the tissue and denature the proteins in adjacent tissue cells, thereby providing a coagulative effect with relatively little thermal spread. In some versions, an electrical current may also be provided through ultrasonic blade (142) and/or clamp pad (146) to also seal the tissue.

An operator may activate buttons (126) to selectively activate transducer assembly (112) to thereby activate ultrasonic blade (142). In the present example, two buttons (126) are provided-one for activating ultrasonic blade (142) at a low power and another for activating ultrasonic blade (142) at a high power. However, it should be understood that any other suitable number of buttons and/or otherwise selectable power levels may be provided. For instance, a foot pedal may be provided to selectively activate transducer assembly (112). Buttons (126) of the present example are positioned such that an operator may readily fully operate instrument (100) with a single hand. For instance, the operator may position their thumb in thumb grip ring (154), position their ring finger in finger grip ring (124), position their middle finger about body (122), and manipulate buttons (126) using their index finger. Of course, any other suitable techniques may be used to grip and operate instrument (100); and buttons (126) may be located at any other suitable positions.

The foregoing components and operabilities of instrument (100) are merely illustrative. Instrument (100) may be configured in numerous other ways as will be apparent to those of ordinary skill in the art in view of the teachings herein. By way of example only, at least part of instrument (100) may be constructed and/or operable in accordance with at least some of the teachings of any of the following, the disclosures of which are all incorporated by reference herein: U.S. Pat. Nos. 5,322,055; 5,873,873; 5,980,510; 6,325,811; 6,783,524; U.S. Pub. No. 2006/0079874; U.S. Pub. No. 2007/0191713; U.S. Pub. No. 2007/0282333; U.S. Pub. No. 2008/0200940; U.S. Pub. No. 2010/0069940; U.S. Pub. No. 2011/0015660; U.S. Pub. No. 2012/0112687; U.S. Pub. No. 2012/0116265; U.S. Pub. No. 2014/0005701; U.S. Pub. No. 2014/0114334; and/or U.S. patent application Ser. No. 14/031,665. Additional merely illustrative variations for instrument (100) will be described in greater detail below. It should be understood that the below described variations may be readily applied to instrument (100) described above and any of the instruments referred to in any of the references that are cited herein, among others.

II. Surgical Instrument with Integrated Tissue Stop

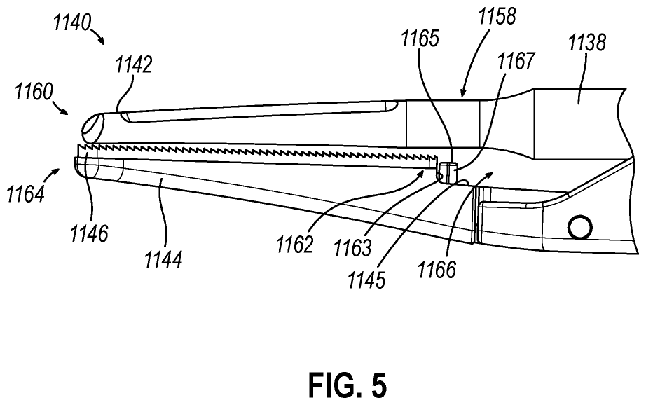

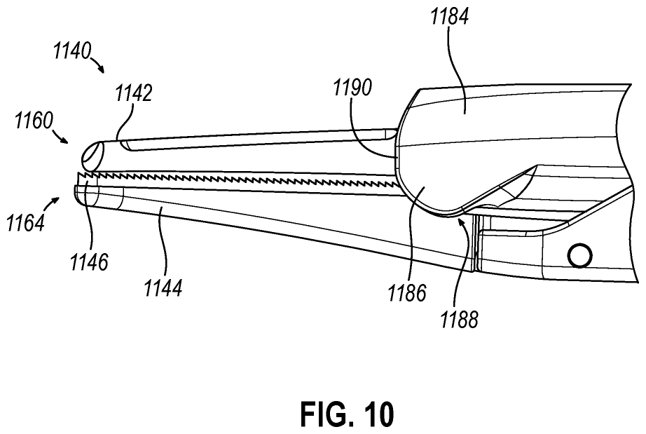

FIGS. 5-12 show an exemplary alternative end effector (1140) that may be used in place of end effector (140). End effector (1140) of this example is substantially similar to end effector (140) described above. In particular, end effector (1140) includes an ultrasonic blade (1142) extending from a guide (1138), a clamp arm (1144), and a clamp pad (1146). Ultrasonic blade (1142), clamp arm (1144), and clamp pad (1146) of this example are substantially similar to ultrasonic blade (142), clamp arm (144), and clamp pad (146), respectively. Guide (1138) may also encompass a guide style element, similar to acoustic waveguide (138), a distal pipe, or any other element generally holding, guiding, or projecting ultrasonic blade (1142) outwardly towards an engagement with clamp arm (144) and clamp pad (1146).

As shown in FIG. 5, ultrasonic blade (1142) distally projects relative to guide (1138). Ultrasonic blade (1142) extends from a proximal end (1158) outwardly away from guide (1138) to terminate at a distal end (1160). Similarly, clamp pad (1146) extends from a proximal end (1162) to a distal end (1164). Proximal end (1158) of ultrasonic blade (1142) and proximal end (1162) of clamp pad (1146) are both disposed generally proximate guide (1138).

A proximal clamp pad surface (1165) of clamp pad (1146) is exposed at proximal end (1162), generally facing guide (1138). Similarly, a proximal clamp arm surface (1163) is generally facing guide (1138) and in some versions of end effector (1140) sized to generally align with proximal clamp pad surface (1165). In some versions of end effector (1140), a pad retention feature (1167) is provided as a projection located at proximate end (1162) of clamp pad (1146) to prevent clamp pad (1146) from sliding proximally off or out of placement with respect to clamp arm (1144). Pad retention feature (1167) is disposed within end effector (1140) to abut proximal clamp pad surface (1165) and thereby prevent displacement of clamp pad (1146).

Guide (1138), ultrasonic blade (1142), clamp pad (1146), and clamp arm (1144) cooperate to define a gap (1166) when end effector (1140) is in the closed configuration. Clamp arm (1144) includes a gap surface (1145) extending generally orthogonally from gap surface (1165) of clamp pad (1146) and proximal clamp arm surface (1163). Gap (1166) facilitates clearance between acoustic waveguide (138) and clamp arm (1144) when end effector (1140) is in a closed configuration.

Users may unintentionally overstuff end effector (1140) with tissue, allowing tissue to extend into gap (1166) and past the length of clamp pad (1146). Tissue in this position may not be effectively cut or sealed due to the lack of clamp pad (1146) to press the tissue into ultrasonic blade (1142). Overstuffing may occur when a user is particularly overzealous when pressing the tissue into the area between clamp pad (1146) and ultrasonic blade (1142) prior to a cut. Alternatively, a user may position the tissue properly initially, yet during the closing stroke, a portion of the tissue may press or slip into gap (1166). Both scenarios may result in an ineffective cut and/or seal, surgical error, or delay.

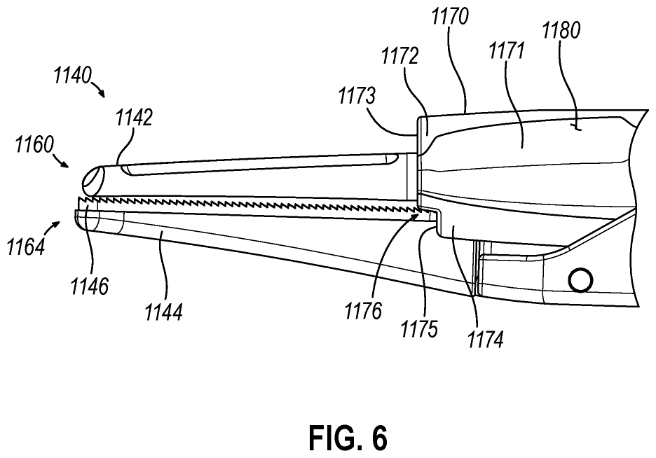

As shown in FIGS. 6-8, a tissue stop (1170) may be provided on or around guide (1138) to prevent tissue from traveling into gap (1166) and overstuffing end effector (1140) with tissue, past the length of ultrasonic blade (1142). Tissue stop (1170) may also be used to provide a visual indication to the user as to where the tissue should be placed within end effector (1140) prior to a closure stroke. Tissue stop (1170) may also be used as an atraumatic heat shield to enable end effector (1140) to self-contain heat from ultrasonic blade (1142) and thereby prevent unintended thermal spread to nearby critical anatomical structures.

Tissue stop (1170) of the present disclosure includes a generally circular or continuous wall (1171). Wall (1171) may be formed into a cone or sheath-like structure around guide (1138), and sized and positioned to prevent tissue from pressing beyond tissue stop (1170) and into gap (1166) and when end effector (1140) moves from the open configuration to the closed configuration with tissue therebetween. In some versions, wall (1171) of tissue stop (1170) may resemble a U-shaped or shroud type of extension off guide (1138) extending over proximal end (1158) of ultrasonic blade (1142). Wall (1171) of tissue stop (1170) may include a profile that abuts either gap surface (1145) of clamp pad (1146), gap surface (1165) of clamp arm (1144), or both, when end effector (1140) is in the closed configuration. Wall (1171) includes an exterior surface (1180) and an interior surface (1182).

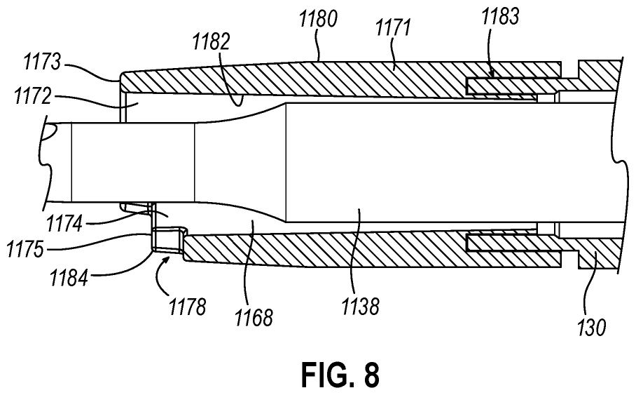

As shown in FIG. 8, tissue stop (1170) defines an interior channel (1168) there through. In some versions of tissue stop (1170), the proximal end (1162) of clamp pad (1146) is disposed in interior channel (1168) when end effector (1140) is in the closed configuration.

Tissue stop (1170) further includes a leading surface. In some versions of tissue stop (1170), the leading surface of tissue stop (1170) is located distally of proximal end (1162) of clamp pad (1146) when end effector (1140) is in the closed configuration. In some versions of tissue stop (1170), the proximal end (1162) of clamp pad (1146) is disposed in interior channel (1168) when end effector (1140) is in the closed configuration.

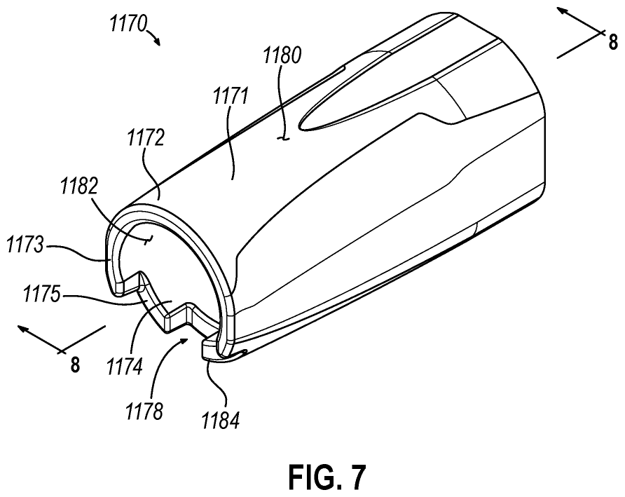

Some versions of wall (1171) of tissue stop (1170) may include a stepped configuration, with a portion of wall (1171) being referred to as a first wall (1172) having a first leading surface (1173) and a portion of wall (1171) being referred to as a second wall (1174) having a second leading surface (1175). The leading surface described above may be embodied in one or both of first leading surface (1173) and second leading surface (1175). First wall (1172) and second wall (1174) define a stepped portion (1176). As shown in FIG. 7, first wall (1172) is sized and positioned to partially encircle ultrasonic blade (1142), with second wall (1174) continuing this partial encirclement. Second wall (1174) defines a notch (1178), wherein notch (1178) sized to receive pad retention feature (1167) therein when end effector (1140) is in the closed configuration.

As shown in FIG. 7, in some versions of tissue stop (1170), a chamfered edge (1184) may be provided at the transition between exterior surface (1180) of wall (1171) and second leading surface (1175) of second wall (1174). Inasmuch as second wall (1174) generally abuts clamp arm (1144) when end effector (1140) is in the closed configuration, chamfered edge (1184) further facilitates tissue stop (1170) pressing onto and abutting clamp arm (1144) as end effector (1140) transitions from the open configuration to the closed configuration.

As shown in FIG. 8, wall (1171) of tissue stop (1170) defines a pocket (1183) therein. Pocket (1183) is sized to receive a portion of another element of instrument (100) for locking tissue stop (1170) in place. For example, a portion of shaft assembly (130) may extend into pocket (1183) to lock tissue stop (1170) in place. In some versions of tissue stop (1170), guide (1138) is entirely encircled by wall (1171) of tissue stop (1170) without another feature or element of end effector (1140) abutting guide (1138) within tissue stop (1170). Thus, guide (1138) passes through tissue stop (1170) free of an abutment therein.

In some other versions of tissue stop (1170), tissue stop (1170) may be over-molded onto guide (1138), or an acoustic waveguide used in place or in conjunction with guide (1138), or on cap (134) to provide a snug fit and additional friction to secure tissue stop (1170) into end effector (1140). Over-molding may also reduce costs and provide the desired ability to fine-tune the tolerances and shape to facilitate first wall (1172) and second wall (1174) fitting as desired with respect to distal end (1164) of clamp pad (1146).

Broadly speaking, first wall (1172), second wall (1174), and notch (1178) are sized and configured to fit complementarily with the profile formed by proximal clamp arm surface (1163), proximal clamp pad surface (1165), gap surface (1145), and pad retention feature (1167). As end effector (1140) moves from the open configuration to the closed configuration with tissue therebetween, second wall (1174) moves into an abutment with clamp arm (1144). As the closing stroke continues, second wall (1174) continues into alignment and first wall (1172) moves into a backstop style orientation with respect to clamp pad (1146), with first leading surface (1173) facing and stopping any tissue that would be moving in a proximal direction toward gap (1166). Gap (1166) is thereby closed and tissue cannot extend beyond clamp pad (1146), ensuring all tissue disposed between ultrasonic blade (1142) and clamp arm (1144) is sealed and/or cut as desired. Further, tissue stop (1170) may provide a heat shield with respect to ultrasonic blade (1142) to prevent unintended thermal spread to nearby tissue or anatomical structures.

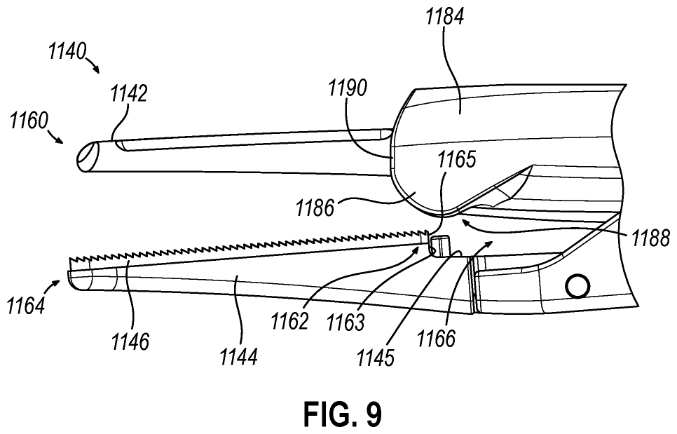

Other versions of the described tissue stop (1170) may have different profiles or features. As shown in FIGS. 9-12, a second exemplary tissue stop, referred to as a tissue stop (1184), includes a pair of deflectable flaps (1186). In some versions of tissue stop (1184), deflectable flaps (1186) and wall (1171) are formed from the same material in and include generally the same thickness throughout the length of wall (1171). In other versions of tissue stop (1184), deflectable flaps (1186) are formed from the same material as the rest of wall (1171), but with a reduced thickness to allow additional flexibility in deflectable flaps (1186) as these elements pass or cam over clamp arm (1144) as end effector (1140) proceeds through its closing stroke to move into the closed configuration.

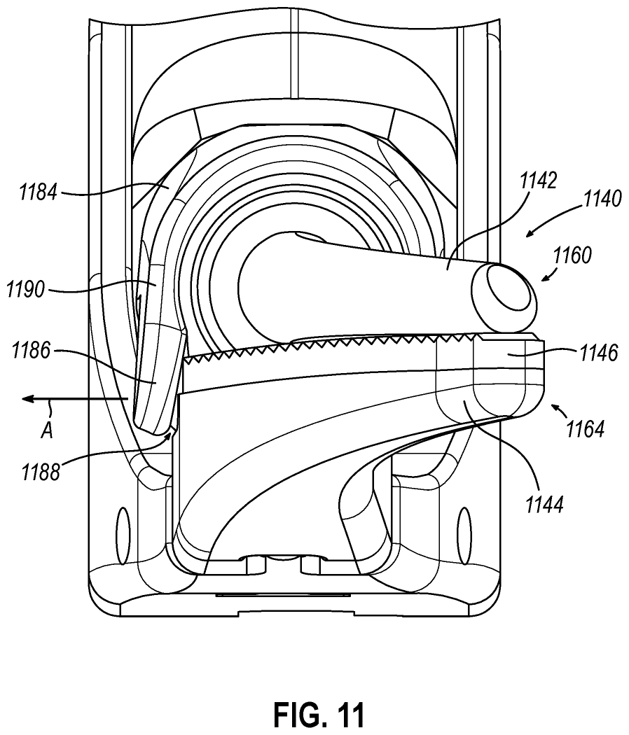

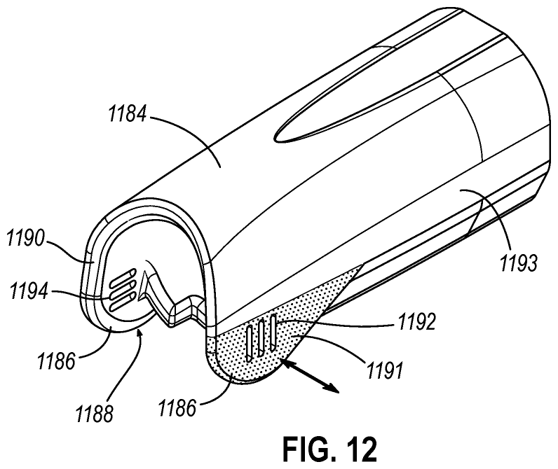

As shown in FIG. 11, deflectable flaps (1186) may further include an interior chamfered area (1188) to aid in camming or slidably abutting deflectable flaps (1186) over clamp pad (1146) and clamp arm (1144) in the direction of Arrow A. As shown in FIG. 12, other versions of deflectable flaps (1186) may include a different material from that which the rest of wall (1171) is comprised. For example, deflectable flaps (1186) may be formed from a first material (1191), while the remainder of tissue stop (1184) may be formed from a second material (1193), whereby first material (1191) is more flexible than second material (1193). Still other versions of delectable flaps (1186) may define one or more slits (1192) extending entirely through deflectable flaps (1186) to allow for more flexibility of each deflectable flap (1186) during the closing stroke. Deflectable flaps (1186) may also include one or more cut out portions (1194) to aid in flexibility, wherein cut out portions (1194) are not entirely cut through deflectable flaps (1186), but a portion of the material is removed to reduce stiffness. Slits (1192) and/or cut out portions (1194) may be curved or s-shaped or may be generally parallel with clamp pad (1146) while in the closed configuration to allow for greater flexibility in the deflection of deflectable flaps (1186). Deflectable flaps (1186) may incorporate atraumatic geometry such as smooth curves should inadvertent tissue contact occur.

Deflectable flaps (1186) may be formed such that a portion of deflectable flaps (1186) remain distal of proximal end (1162) of clamp pad (1146) through the range of motion between the open configuration and the closed configuration of end effector (1140).

Similar to tissue stop (1170), once tissue stop (1184) moves into the closed configuration, deflectable flaps (1186) cam or otherwise slide or travel over the portion of clamp arm (1144) proximate proximal end (1162) of clamp pad (1146). This provides for containing heat from ultrasonic blade (1142) as well and reducing inadvertent overstuffing of end effector (1140) with tissue. As shown in FIG. 11, deflectable flaps (1186) may include a slight interference fit with clamp arm (1144), which allows deflectable flaps (1186) to deflect out of the way as clamp arm (1144) and ultrasonic blade (1142) move through its closure stroke.

The length of deflectable flaps (1186) determine how long these elements will act as a tissue stop relative to the open and close stroke of clamp arm (1144). Extending the length of deflectable flaps (1186) in turn extends the time those elements act as a tissue stopping device. In some versions of tissue stop (1184), deflectable flaps (1186) include a leading surface (1190). Leading surface (1190) may be formed with a particular radius. In some version of leading surface (1190), the radius is configured to match proximal end (1162) of clamp pad (1146). As deflectable flaps (1186) rise during an open stroke, the radius of leading surface (1190) ensures deflectable flaps (1186) do not travel past proximal end (1162) of clamp pad (1146) to maintain the tissue stopping functionality. Further this radius on leading surface (1190) and smooth curvature on other surfaces or deflectable flaps (1186) provide atraumatic geometry should any tissue inadvertently contact tissue stop (1184).

Similar to tissue stop (1170), leading surface (1190) of tissue stop (1184) is located distally of proximal end (1162) of clamp pad (1146) when end effector (1140) is in the closed configuration.

In operation, as a user actuates end effector (1140) to move ultrasonic blade (1142) and clamp arm (1144) from the open configuration into the closed configuration, deflectable flaps (1186) travel past gap (1166) and extend over the portion of clamp arm (1144) proximate proximal end (1162) of clamp pad (1146). This enables an atraumatic mechanism that will prevent undesired tissue damage due to overstuffing as well as providing a visual indicator to the user as to where tissue should be placed within end effector (1140) with respect to clamp pad (1146).

Some versions of tissue stop (1170, 1184) are shaped to abut clamp arm (1144) when end effector (1140) is in the closed configuration. Some versions of tissue stop (1170, 1184) are shaped to abut clamp pad (1146) when end effector (1140) is in the closed configuration. Some versions of tissue stop (1170, 1184) are shaped to abut gap surface (1145) when the end effector is in the closed configuration. Some versions of tissue stop (1170, 1184) are shaped to abut proximal clamp arm surface (1163) when the end effector is in the closed configuration. Some versions of tissue stop (1170, 1184) are shaped to abut proximal clamp pad surface (1165) when the end effector is in the closed configuration.

In some versions of tissue stop (1170, 1184), an elastomeric material is used. With respect to flexibility, in some versions of tissue stop (1170, 1184), the elastomeric material may measure between 30 and 90 (inclusive) on the Shore A hardness scale.

With respect to assembly of ultrasonic surgical instrument (100), passing ultrasonic blade (1142) through a distal pipe element requires an amount of force sufficient to overcome the friction between these two elements when distal pipe is formed from an elastomer. To reduce friction, distal pipe may be lubricated prior to assembly. This pre-assembly lubrication may utilize medical grade lubricants such as sodium stearate or silicone fluid. Alternatively, distal pipe may be formed from a self-lubricating elastomer such as Nusil MED1-4855 Liquid silicone rubber. Nusil MED1-4855 Liquid silicone rubber is a self-lubricating injection moldable elastomer with high lubrication properties and is suitable for over-molding.

### III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical instrument, comprising: (a) a shaft; and (b) an end effector located at a distal end of the shaft, wherein the end effector is selectively movable between an open configuration and a closed configuration, wherein the end effector comprises: (i) a guide, (ii) an ultrasonic blade extending distally from the guide and operable to treat tissue with ultrasonic energy, (iii) a clamp arm movable relative to the ultrasonic blade, (iv) a clamp pad disposed on the clamp arm and extending from a proximal end to a distal end, (v) a tissue stop encircling the guide, wherein the tissue stop includes a leading surface, wherein the leading surface of the tissue stop is located distally of the proximal end of the clamp pad when the end effector is in the closed configuration.

### Example 2

The surgical instrument of Example 1, wherein the tissue stop abuts the clamp arm when the end effector is in the closed configuration.

### Example 3

The surgical instrument of any one or more of Examples 1 through 2, wherein the clamp arm includes a gap surface, wherein the tissue stop abuts the gap surface when the end effector is in the closed configuration.

### Example 4

The surgical instrument of any one or more of Examples 1 through 3, wherein the clamp arm includes a proximal clamp arm surface, wherein the tissue stop abuts the proximal clamp arm surface when the end effector is in the closed configuration.

### Example 5

The surgical instrument of any one or more of Examples 1 through 4, wherein the clamp pad includes proximal clamp pad surface, wherein the tissue stop abuts the proximal clamp pad surface when the end effector is in the closed configuration.

### Example 6

The surgical instrument of any one or more of Examples 1 through 5, wherein the tissue stop includes a first portion and a second portion, wherein the second portion extends from the first portion, wherein the first portion extends entirely around the ultrasonic blade, wherein the second portion extends partially around the ultrasonic blade.

### Example 7

The surgical instrument of any one or more of Examples 1 through 6, wherein the second portion includes a U-shaped profile.

### Example 8

The surgical instrument of any one or more of Examples 1 through 7, wherein the end effector comprises further comprises: (a) a pad retention feature proximate the clamp pad, wherein the pad retention feature is configured to prevent the clamp pad from sliding proximally therebeyond; and (b) a notch defined by the tissue stop, wherein a portion of the pad retention feature is disposed within the notch when the end effector is in the closed configuration.

### Example 9

The surgical instrument of any one or more of Examples 1 through 8, wherein the tissue stop includes a pair of deflectable flaps, wherein the ultrasonic blade is disposed between the pair of deflectable flaps.

### Example 10

The surgical instrument of any one or more of Examples 1 through 9, wherein the pair of deflectable flaps abut the clamp arm when the end effector is in the closed configuration.

### Example 11

The surgical instrument of any one or more of Examples 1 through 10, wherein the pair of deflectable flaps are formed from a first material, wherein the remainder of the tissue stop is formed from a second material.

### Example 12

The surgical instrument of any one or more of Examples 1 through 11, wherein at least one of the pair of the deflectable flaps defines one of a slit therethrough and a cut out portion.

### Example 13

The surgical instrument of any one or more of Examples 1 through 12, wherein at least one of the pair of deflectable flaps includes a first thickness, wherein the remainder of the tissue stop includes a second thickness, wherein the first thickness is less than a second thickness.

### Example 14

The surgical instrument of any one or more of Examples 1 through 13, wherein the tissue stop includes a deflectable flap, wherein a portion of the deflectable flap remains distal of the proximal end of the clamp pad through the range of motion between the open configuration and the closed configuration.

### Example 15

The surgical instrument of any one or more of Examples 1 through 14, wherein the pair of deflectable flaps abut the clamp arm throughout the entire range of motion between the open configuration and the closed configuration.

### Example 16

A surgical instrument, comprising: (a) a shaft; and (b) an end effector located at a distal end of the shaft, wherein the end effector is selectively movable between an open configuration and a closed configuration, wherein the end effector comprises: (i) an ultrasonic blade extending from a guide and operable to treat tissue with ultrasonic energy, (ii) a guide, wherein the ultrasonic blade extends distally from the guide, (iii) a clamp arm movable relative to the ultrasonic blade, (iv) a clamp pad disposed on the clamp arm and extending from a proximal end to a distal end, (v) a tissue stop defining an interior channel, wherein the guide is disposed in the interior channel, wherein the proximal end of the clamp pad is disposed in the interior channel when the end effector is in the closed configuration.

### Example 17

The surgical instrument of any one or more of Example 16, wherein the end effector comprises further comprises: (a) a pad retention feature proximate the clamp pad, wherein the pad retention feature is configured to prevent the clamp pad from sliding proximally therebeyond; and (b) a notch defined by the tissue stop, wherein a portion of the pad retention feature is disposed within the notch when the end effector is in the closed configuration.

### Example 18

The surgical instrument of any one or more of Examples 16 or 17, herein the tissue stop includes a pair of deflectable flaps, wherein the ultrasonic blade is disposed between the pair of deflectable flaps, wherein the pair of deflectable flaps abut the clamp arm when the end effector is in the closed configuration.

### Example 19

The surgical instrument of any one or more of Examples 16 through 18, wherein the pair of deflectable flaps are formed from a first material, wherein the remainder of the tissue stop is formed from a second material.

### Example 20

A surgical instrument configured to apply ultrasonic energy to tissue disposed therein, the surgical instrument comprising: (a) a shaft; and (b) an end effector located at a distal end of the shaft, wherein the end effector is selectively movable between an open configuration and a closed configuration, wherein the end effector comprises: (i) an ultrasonic blade extending from a guide and operable to treat tissue with ultrasonic energy, (ii) a guide, wherein the ultrasonic blade extends distally from the guide, (iii) a clamp arm movable relative to the ultrasonic blade, (iv) a clamp pad disposed on the clamp arm and extending from a proximal end to a distal end, (v) a pad retention feature proximate the clamp pad, wherein the pad retention feature is configured to prevent the clamp pad from sliding proximally therebeyond, (vi) a tissue stop encircling the guide, (vii) a notch defined by the tissue stop, wherein a portion of the pad retention feature is disposed within the notch when the end effector is in the closed configuration, wherein the tissue stop is configured to prevent tissue from moving proximally past the clamp pad when the end effector moves into the closed configuration, wherein the tissue stop abuts the clamp arm when the end effector is in the closed configuration, wherein the tissue stop includes a first portion and a second portion, wherein the second portion extends from the first portion, wherein the first portion extends entirely around the ultrasonic blade, wherein the second portion extends partially around the ultrasonic blade, and wherein the second portion includes a U-shaped profile.

### IV. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of any claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, California. Similarly, those of ordinary skill in the art will recognize that various teachings herein may be readily combined with various teachings of any of the following: U.S. Pat. No. 5,792,135, entitled “Articulated Surgical Instrument For Performing Minimally Invasive Surgery With Enhanced Dexterity and Sensitivity,” issued Aug. 11, 1998, the disclosure of which is incorporated by reference herein; U.S. Pat. No. 5,817,084, entitled “Remote Center Positioning Device with Flexible Drive,” issued Oct. 6, 1998, the disclosure of which is incorporated by reference herein; U.S. Pat. No. 5,878,193, entitled “Automated Endoscope System for Optimal Positioning,” issued Mar. 2, 1999, the disclosure of which is incorporated by reference herein; U.S. Pat. No. 6,231,565, entitled “Robotic Arm DLUS for Performing Surgical Tasks,” issued May 15, 2001, the disclosure of which is incorporated by reference herein; U.S. Pat. No. 6,783,524, entitled “Robotic Surgical Tool with Ultrasound Cauterizing and Cutting Instrument,” issued Aug. 31, 2004, the disclosure of which is incorporated by reference herein; U.S. Pat. No. 6,364,888, entitled “Alignment of Master and Slave in a Minimally Invasive Surgical Apparatus,” issued Apr. 2, 2002, the disclosure of which is incorporated by reference herein; U.S. Pat. No. 7,524,320, entitled “Mechanical Actuator Interface System for Robotic Surgical Tools,” issued Apr. 28, 2009, the disclosure of which is incorporated by reference herein; U.S. Pat. No. 7,691,098, entitled “Platform Link Wrist Mechanism,” issued Apr. 6, 2010, the disclosure of which is incorporated by reference herein; U.S. Pat. No. 7,806,891, entitled “Repositioning and Reorientation of Master/Slave Relationship in Minimally Invasive Telesurgery,” issued Oct. 5, 2010, the disclosure of which is incorporated by reference herein; U.S. Pat. No. 8,844,789, entitled “Automated End Effector Component Reloading System for Use with a Robotic System,” issued Sep. 30, 2014, the disclosure of which is incorporated by reference herein; U.S. Pat. No. 8,820,605, entitled “Robotically-Controlled Surgical Instruments,” issued Sep. 2, 2014, the disclosure of which is incorporated by reference herein; U.S. Pat. No. 8,616,431, entitled “Shiftable Drive Interface for Robotically-Controlled Surgical Tool,” issued Dec. 31, 2013, the disclosure of which is incorporated by reference herein; U.S. Pat. No. 8,573,461, entitled “Surgical Stapling Instruments with Cam-Driven Staple Deployment Arrangements,” issued Nov. 5, 2013, the disclosure of which is incorporated by reference herein; U.S. Pat. No. 8,602,288, entitled “Robotically-Controlled Motorized Surgical End Effector System with Rotary Actuated Closure Systems Having Variable Actuation Speeds,” issued Dec. 10, 2013, the disclosure of which is incorporated by reference herein; U.S. Pat. No. 9,301,759, entitled “Robotically-Controlled Surgical Instrument with Selectively Articulatable End Effector,” issued Apr. 5, 2016, the disclosure of which is incorporated by reference herein; U.S. Pat. No. 8,783,541, entitled “Robotically-Controlled Surgical End Effector System,” issued Jul. 22, 2014, the disclosure of which is incorporated by reference herein; U.S. Pat. No. 8,479,969, entitled “Drive Interface for Operably Coupling a Manipulatable Surgical Tool to a Robot,” issued Jul. 9, 2013; U.S. Pat. No. 8,800,838, entitled “Robotically-Controlled Cable-Based Surgical End Effectors,” issued Aug. 12, 2014, the disclosure of which is incorporated by reference herein; and/or U.S. Pat. No. 8,573,465, entitled “Robotically-Controlled Surgical End Effector System with Rotary Actuated Closure Systems,” issued Nov. 5, 2013, the disclosure of which is incorporated by reference herein.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a clinician immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention is understood not to be limited to the details of structure and operation shown and described in the specification and drawings.

I/We claim:

1. A surgical instrument, comprising:
(a) a shaft; and
(b) an end effector located at a distal end of the shaft, wherein the end effector is selectively movable between an open configuration and a closed configuration, wherein the end effector comprises:
(i) a guide,
(ii) an ultrasonic blade extending distally from the guide and operable to treat tissue with ultrasonic energy,
(iii) a clamp arm movable relative to the ultrasonic blade,
(iv) a clamp pad disposed on the clamp arm and extending from a proximal end to a distal end,
(v) a tissue stop encircling the guide, wherein the tissue stop includes a leading surface,
wherein the leading surface of the tissue stop is located distally of the proximal end of the clamp pad when the end effector is in the closed configuration.

2. The surgical instrument of claim 1, wherein the tissue stop abuts the clamp arm when the end effector is in the closed configuration.

3. The surgical instrument of claim 1, wherein the clamp arm includes a gap surface, wherein the tissue stop abuts the gap surface when the end effector is in the closed configuration.

4. The surgical instrument of claim 1, wherein the clamp arm includes a proximal clamp arm surface, wherein the tissue stop abuts the proximal clamp arm surface when the end effector is in the closed configuration.

5. The surgical instrument of claim 1, wherein the clamp pad includes proximal clamp pad surface, wherein the tissue stop abuts the proximal clamp pad surface when the end effector is in the closed configuration.

6. The surgical instrument of claim 1, wherein the tissue stop includes a first portion and a second portion, wherein the second portion extends from the first portion, wherein the first portion extends entirely around the ultrasonic blade, wherein the second portion extends partially around the ultrasonic blade.

7. The surgical instrument of claim 6, wherein the second portion includes a U-shaped profile.

8. The surgical instrument of claim 1, wherein the end effector comprises further comprises:
(a) a pad retention feature proximate the clamp pad, wherein the pad retention feature is configured to prevent the clamp pad from sliding proximally therebeyond; and
(b) a notch defined by the tissue stop, wherein a portion of the pad retention feature is disposed within the notch when the end effector is in the closed configuration.

9. The surgical instrument of claim 1, wherein the tissue stop includes a pair of deflectable flaps, wherein the ultrasonic blade is disposed between the pair of deflectable flaps.

10. The surgical instrument of claim 9, wherein the pair of deflectable flaps abut the clamp arm when the end effector is in the closed configuration.

11. The surgical instrument of claim 9, wherein the pair of deflectable flaps are formed from a first material, wherein a remainder of the tissue stop is formed from a second material.

12. The surgical instrument of claim 9, wherein at least one of the pair of deflectable flaps defines one of a slit therethrough and a cut out portion.

13. The surgical instrument of claim 9, wherein at least one of the pair of deflectable flaps includes a first thickness, wherein a remainder of the tissue stop includes a second thickness, wherein the first thickness is less than a second thickness.

14. The surgical instrument of claim 9, wherein the tissue stop includes a deflectable flap, wherein a portion of the deflectable flap remains distal of the proximal end of the clamp pad as the end effector moves between the open configuration and the closed configuration.

15. The surgical instrument of claim 9, wherein the pair of deflectable flaps abut the clamp arm as the end effector moves between the open configuration and the closed configuration.

16. A surgical instrument, comprising:
(a) a shaft; and
(b) an end effector located at a distal end of the shaft, wherein the end effector is selectively movable between an open configuration and a closed configuration, wherein the end effector comprises:
(i) an ultrasonic blade extending from a guide and operable to treat tissue with ultrasonic energy,
(ii) a guide, wherein the ultrasonic blade extends distally from the guide,
(iii) a clamp arm movable relative to the ultrasonic blade,
(iv) a clamp pad disposed on the clamp arm and extending from a proximal end to a distal end,
(v) a tissue stop defining an interior channel, wherein the guide is disposed in the interior channel,
wherein the proximal end of the clamp pad is disposed in the interior channel when the end effector is in the closed configuration.

17. The surgical instrument of claim 16, wherein the end effector comprises further comprises:
(a) a pad retention feature proximate the clamp pad, wherein the pad retention feature is configured to prevent the clamp pad from sliding proximally therebeyond; and
(b) a notch defined by the tissue stop, wherein a portion of the pad retention feature is disposed within the notch when the end effector is in the closed configuration.

18. The surgical instrument of claim 16, wherein the tissue stop includes a pair of deflectable flaps, wherein the ultrasonic blade is disposed between the pair of deflectable flaps, wherein the pair of deflectable flaps abut the clamp arm when the end effector is in the closed configuration.

19. The surgical instrument of claim 18, wherein the pair of deflectable flaps are formed from a first material, wherein a remainder of the tissue stop is formed from a second material.

20. A surgical instrument configured to apply ultrasonic energy to tissue disposed therein, the surgical instrument comprising:
(a) a shaft; and
(b) an end effector located at a distal end of the shaft, wherein the end effector is selectively movable between an open configuration and a closed configuration, wherein the end effector comprises:
(i) an ultrasonic blade extending from a guide and operable to treat tissue with ultrasonic energy,
(ii) a guide, wherein the ultrasonic blade extends distally from the guide,
(iii) a clamp arm movable relative to the ultrasonic blade,
(iv) a clamp pad disposed on the clamp arm and extending from a proximal end to a distal end,
(v) a pad retention feature proximate the clamp pad, wherein the pad retention feature is configured to prevent the clamp pad from sliding proximally therebeyond,
(vi) a tissue stop encircling the guide,
(vii) a notch defined by the tissue stop, wherein a portion of the pad retention feature is disposed within the notch when the end effector is in the closed configuration,
wherein the tissue stop is configured to prevent tissue from moving proximally past the clamp pad when the end effector moves into the closed configuration,
wherein the tissue stop abuts the clamp arm when the end effector is in the closed configuration,
wherein the tissue stop includes a first portion and a second portion, wherein the second portion extends from the first portion, wherein the first portion extends entirely around the ultrasonic blade, wherein the second portion extends partially around the ultrasonic blade, and
wherein the second portion includes a U-shaped profile.
